(19) United States
(12) Patent Application Publication
(10) Pub. No.: US 2002/0132342 A1
(43) Pub. Date: Sep. 19, 2002

(54) Method for generating immortal dendritic cell lines

(76) Inventors: David Ronald Fitzpatrick, Seattle, WA (US); Eileen Rose Roux, Seattle, WA (US); Charles Richard Maliszewski, Seattle, WA (US); Jacques Jean Peschon, Seattle, WA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/013,587

(22) Filed: Dec. 10, 2001

Related U.S. Application Data
(60) Provisional application No. 60/256,502, filed on Dec. 13, 2000.
(60) Provisional application No. 60/275,243, filed on Mar. 9, 2001.

(51) Int. Cl. C12N 5/08; C12N 15/87

(52) U.S. Cl. 435/372; 435/455

(57) ABSTRACT

Immortalized mammalian dendritic cell lines are provided that have characteristics of functional, mature dendritic cells; defined by surface phenotype, constitutive and inducible gene expression patterns, and functional effects on T lymphocytes. The cell lines are capable of continuous in vitro proliferation for extended periods of time. Of particular interest is the ability of the cell lines to present antigen in a stimulatory manner to naive T cells. The dendritic cell lines are useful in gene discovery, the generation of dendritic cell subset-specific antibodies and probes; defining stages of dendritic cell differentiation and maturation; for induction of antigen specific stimulation of lymphocytes; for dissection of antigen specific anergy or tolerance in lymphocytes; for characterization of dendritic cell-natural killer cell interactions; for in vitro and in vivo analyses of dendritic cell-endothelial cell interactions; and for screening assays.

A key component in the mammalian immune response is the dendritic cell (DC), which processes and presents antigens to lymphocytes. Dendritic cells are believed to originate from hematopoietic stem cells in the bone marrow. As the cells differentiate, they migrate to organs and peripheral tissues, and undergo additional differentiation. These immature dendritic cells are inefficient at antigen presentation, express low levels of MHC Class II molecules and do not stimulate proliferation of T-cells in an allogeneic mixed leukocyte reaction. On exposure to antigen, the immature cells differentiate to functional antigen presenting cells, and migrate to lymph nodes and spleen. The mature dendritic cells exhibit high levels of MHC Class II, accessory and co-stimulatory molecules.

Although they are scarce, dendritic cells are of great interest because of their unique potency in antigen presentation. Only dendritic cells are capable of priming naïve T lymphocytes, and eliciting generation of cytotoxic T cells to soluble antigens. Because of their scarcity, culture methods to generate DC from progenitor cells have been developed. DC at different stages of maturation, based on phenotype and capacity to capture antigen, can be obtained depending on culture conditions. These methods have generally relied on the isolation of progenitor cells from bone marrow, cord blood, adult blood CD34+ progenitors, etc., followed by culture in vitro with antigen, and/or one or more growth factors. Factors that have been tested include Flt3 ligand, GM-CSF, IL-4, stem cell factor, and TNF-α, among others.

A key molecule in the maturation and activation of dendritic cells is Flt3 ligand (Flt3-L). In vivo administration of Flt3-L to mice results in preferential mobilization or release of DC precursors from the bone marrow to the periphery and into lymphoid organs, and can increase the number of circulating DC 10-30 fold (Maraskovsky et al. (1996) J. Exp. Med. 184:1953-1962). It has also been shown to have a similar effect in the expansion of functionally competent human DC in vivo (Maraskovsky et al. (2000) Blood 96(3):878-84). The addition of GM-CSF further increases the yield of functional DC (Avigan et al. (1999) Clinical Cancer Research 5(10):2735-41).

Cells that can be continuously cultured are known as immortalized cells. Mammalian diploid cells have a limited life span, ending in replicative senescence, in contrast to cell lines derived from tumors, which show an indefinite life span and are immortal. Immortalized cells have advantages over non-immortalized cells because they can be cultured to provide large numbers of uniform cell populations. Immortalized cells are routinely used for understanding intracellular activities, such as the replication and transcription of DNA, metabolic processes and drug metabolism. Immortalized cells are also useful in examining specific cell-cell interactions, such as antigen presentation. However, many cell types have remained recalcitrant to isolation and continuous culture. In addition, many cells lose some of their differentiated properties during culture. Dendritic cells are an example of a cell type that has been difficult to immortalize.

In some instances, cells have been immortalized through the introduction of oncogenes. For example, simian virus 40 (SV40), a papovavirus of rhesus macaque origin, is a potent DNA tumor virus that induces tumors in rodents and transforms many types of cells in culture, including those of human origin. This virus has been a favored laboratory model for mechanistic studies of molecular processes in eukaryotic cells and of cellular transformation. The viral replication protein, named large T antigen (T-ag), is also the viral oncoprotein.

Transgenic mice have been used to study the process of SV40-induced oncogenesis in a broad range of tissues including mammary gland, salivary gland, pancreas, prostate, liver, lung, kidney, intestine, brain, choroid plexus, lens of the eye, bone, smooth muscle and cartilage (see review by Furth (1998) Dev Biol Stand 94:281-7). The focus of these studies rests primarily on the action of the major transforming viral oncoprotein, the large T antigen (T-ag).

The importance of dendritic cells in coordinating immune responsiveness makes them of great interest for study, both in vivo and with in vitro cultures. The present invention addresses this issue.

Literature

Gasperi et al. (1999) J. Leukocyte Biol. 66:263:267 discuss retroviral gene transfer, rapid selection, and maintenance of the immature phenotype in mouse dendritic cells. Volkmann et al. (1996) Eur. J. Immunol. 26:2565 describe a conditionally immortalized dendritic cell line.

Steinman et al. (1999) U.S. Pat. No. 5,994,126 provide methods for the in vitro proliferation of dendritic cell precursors with GM-CSF, IL4, and/or TNFα. Talmor et al. (1998) Eur. J. Immunol. 28:811 also expand cells in vitro with cocktails of growth factors, as do Winzler et al. (1997) J. Exp. Med. 185:317. Girolomoni et al. (1995) Eur. J. Immunol. 25:2163 discuss the establishment of a cell line with features of early dendritic cell precursors from fetal mouse skin. Fairchild et al. (2000) Curr. Biol. 10:1515-1518 describe the directed differentiation of dendritic cells from mouse embryonic stem cells.

Yamada and Katz (1999) J. Immunol. 163:5331 generate dendritic cells from a CD14⁺ cell line with IL-4, TNF-α, IL-1β, and agonistic anti-CD40 monoclonal antibody. Paglia et al. (1993). J. Exp. Med. 178:1893 discuss immortalized stem cell lines.

Other relevant patents include Gopal et al. (1998) U.S. Pat. No. 5,811,297; and Moore (1998) U.S. Pat. No. 5,830,682; and MacKay and Moore (1997), U.S. Pat. No. 5,648,219.

### SUMMARY OF THE INVENTION

Methods and cell lines are provided for the generation of immortalized mammalian dendritic cell lines. The dendritic cell lines have characteristics of functional, mature dendritic cells as defined by surface phenotype, constitutive and inducible gene expression patterns, functional effects on CD8⁺ and/or CD4⁺ T lymphocytes, secretion of cytokines, and are further capable of continuous in vitro proliferation for extended periods of time. Of particular interest is the ability of the cell lines to present antigen in a stimulatory manner to naïve T cells. The dendritic cell lines are useful in molecule discovery, using DNA-, RNA- and protein-based approaches; the generation of dendritic cell subset-specific antibodies and probes; defining stages of dendritic cell differentiation and maturation; for induction of antigen specific stimulation of T and/or B lymphocytes; for dissection of antigen specific anergy or tolerance in T and/or B lymphocytes; for characterization of dendritic cell-natural killer cell interactions; for in vitro and in vivo analysis of dendritic cell-endothelial cell interactions; and for screening assays to determine the effect of biological response modifiers on dendritic cells.

### BRIEF DESCRIPTION OF THE DRAWINGS

FIG. 1 is a map of the SV40 containing vector used in generating transgenic mice.

FIG. 2 is a flow chart depicting an example of timing and combinations of cytokine supplements.

FIGS. 3A and 3B illustrate examples of cloning frequencies and clonal growth rates, respectively, for dendritic cell lines generated from SV40 transgenic mice.

FIG. 4 is a photograph depicting typical dendritic cell line morphology.

FIGS. 5A, 5B, and 5C are graphs showing induction and expression of IL-10 and IL-12 in dendritic cell lines.

FIGS. 6A, 6B and 6C are graphs depicting the priming of antigen specific in vivo responses with dendritic cell lines.

FIG. 7 illustrates the relative induction of mRNA encoding for the IL-12p35 subunit (left panels) and the IL-12p40 subunit (right panels) in response to stimulation.

### DETAILED DESCRIPTION OF THE EMBODIMENTS

Immortalized mammalian dendritic cell lines are provided, which can have characteristics of functional, mature dendritic cells, including antigen presentation to naïve CD8⁺ and/or CD4⁺ T cells, and appropriate synthesis of cytokines in response to stimulus. The dendritic cell lines find a number of uses, e.g. in developing dendritic cell specific reagents; elucidating patterns of gene expression in dendritic cells at various stages; in antigen presentation; compound screening, and the like.

The dendritic cells of the invention are derived from genetically modified progenitor cells, where the cells express a cell growth-promoting oncogene with expression targeted to dendritic cells via specific control sequences. The genetically altered cells may be provided in the context of a transgenic animal where substantially all cells comprise the genetic modification, or alternatively may be provided as modified stem or progenitor cells. Dendritic cells arising from the genetically modified progenitor cells are expanded in vivo or in vitro by growth factor treatment, then cultured in vitro. The cultured cells are optionally further transformed for one or more of the following: growth factor independence; resistance to cell death; and resistance to cell senescence.

Mammalian species useful as a source for the dendritic cell lines include canines; felines; equines; bovines; ovines; etc. and primates, particularly humans. Animal models, particularly small mammals, e.g. mouse, rat, rabbit, guinea pig, etc. are particularly useful for experimental investigations.

The cell lines of the invention may be passaged for extended periods of time, usually at least about 2 months, more usually at least about 4 months, and may be as long as about 18 months; and may be immortalized. Cells that can be continuously cultured are known as immortalized cells. Mammalian diploid cells have a limited life span, ending in replicative senescence, in contrast to cell lines derived from tumors, which show an indefinite life span and are immortalized. In a preferred embodiment, the dendritic cell lines of the present invention simultaneously maintain both an immortalized phenotype, i.e. they do not enter cell cycle arrest; and/or the ability to perform dendritic cell functions, e.g. the ability to take up antigen, migrate to lymphoid tissues, and/or stimulate naïve T and/or B lymphocytes.

Cells may be stored using conventional methods well known to those ordinarily skilled in the art. For example, cells may be resuspended in growth medium or in serum with 15% dimethylsulfoxide (DMSO) added and frozen at a temperature of −80° C. or lower.

Dendritic cell. As used herein, the term refers to any member of a diverse population of morphologically similar cell types found in lymphoid or non-lymphoid tissues. Dendritic cells are referred to as “professional” antigen presenting cells, and have a high capacity for sensitizing MHC-restricted T cells. Dendritic cells may be recognized by function, by phenotype and/or by gene expression pattern, particularly by cell surface phenotype. These cells are characterized by their distinctive morphology, high levels of surface MHC-class II expression and ability to present antigen to CD4⁺ and/or CD8⁺ T cells, particularly to naïve T cells (Steinman et al.. (1991) Ann. Rev. Immunol. 9:271; incorporated herein by reference for its description of such cells).

The cell surface of dendritic cells is unusual, with characteristic veil-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Rα), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3 TLR4, TLR5, TLR6, TLR9, and several lectins. The patterns of expression of these cell surface markers may vary along with the maturity of the dendritic cells, their tissue of origin, and/or their species of origin. The cell surface phenotype of exemplary cell lines generated by the methods of the invention is provided in Table 1 of the Examples. Cells having specific features of interest, e.g. those having a subset of mature phenotypic markers, etc. may be selected from the cell lines.

Immature dendritic cells express low levels of MHC class II, but are capable of endocytosing antigenic proteins and processing them for presentation in a complex with MHC class II molecules. Activated dendritic cells express high levels of MHC class II, ICAM-1 and CD86, and are capable of stimulating the proliferation of naïve allogeneic T cells, e.g. in a mixed leukocyte reaction (MLR).

Functionally, dendritic cells may be identified by any convenient assay for determination of antigen presentation. Such assays may include testing the ability to stimulate antigen-primed and/or naive T cells by presentation of a test antigen, followed by determination of T cell proliferation, release of IL-2, and the like.

Progenitor cells. Progenitor cells, as used herein, refer to cells that ultimately give rise to dendritic cells. Such cells may include progenitor cells that are dedicated to a single hematopoietic lineage, to hematopoietic stem cells that give rise to all hematopoietic lineages, or to cells such as germ cells or embryonic stem cells that give rise to all cells in the body.

In one embodiment of the invention, the progenitor cells are provided through the generation of a transgenic animal, where essentially all cells in the animal contain the genetic modification of interest. The generation of transgenic animals is well known in the art. Transgenic animals may be made through homologous recombination or through random integration of a vector into the genome. Vectors for stable integration include plasmids, retroviruses and other animal viruses, YACs, and the like.

DNA constructs for homologous recombination will comprise at least a portion of a targeted gene with the desired genetic modification, and will include regions of homology to the target locus. The regions of homology may include coding regions, or may utilize intron and/or genomic sequence. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line may be employed, or embryonic cells may be obtained freshly from a host, e.g. mouse, rat, guinea pig, etc. When ES or embryonic cells have been transformed, they may be used to produce transgenic animals. After transformation, the cells are cultured, and those that have the desired genetic modification used for embryo manipulation and blastocyst injection, to create chimeric animals, and ultimately transgenics. The transgenic animals may be any non-human mammal, such as laboratory animals, domestic animals, etc.

Alternatively, manipulated embryonic stem cells may be cultured in vitro and differentiated into dendritic cells using cytokines such as GM-CSF, Flt3L and/or IL-3 (Fairchild et al. (2000) Curr. Biol. 10:1515).

In an alternative embodiment of the invention, particularly where it is not possible to generate transgenic animals, other progenitor and/or stem cells may be genetically modified. Of particular interest are hematopoietic stem cells for this purpose. Such cells have been shown to be genetically modified by viral transduction and other means in a number of reports (for example, see Allay et al. (1998) Nat Med 4(10):1136-43; Zhao et al. Blood 90(12):4687-98; Chen et al (2000) Stem Cells 18(5):352-9; Guenechea et al. (2000) Mol Ther 1(6):566-73). Introduction of the exogenous DNA may be performed in vitro or in vivo, usually in vitro, and the modified cells then reintroduced into a host animal or maintained in culture.

To prove that one has genetically modified progenitor cells, various techniques may be employed. The genome of the cells may be restricted and used with or without amplification. The polymerase chain reaction; gel electrophoresis; restriction analysis; Southern, Northern, and Western blots; sequencing; or the like, may all be employed. The cells may be grown under various conditions to ensure that the cells are capable of differentiation while maintaining the ability to express the introduced DNA. Various tests in vitro and in vivo may be employed to ensure that the pluripotent capability of the cells has been maintained.

Immunocompromised mammalian hosts suitable for implantation of the genetically modified progenitor cells exist or can be created. The significant factor is that the immunocompromised host is incapable of mounting an immune response against the introduced cells. Of particular interest are small mammals, e.g. rabbits, gerbils, hamsters, guinea pigs, etc., particularly rodents, e.g. mouse and rat, which are immunocompromised due to a genetic defect which results in an inability to undergo germline DNA rearrangement at the loci encoding immunoglobulins and T-cell antigen receptors or to a genetic defect in thymus development (nu/nu).

Presently available hosts include mice that have been genetically engineered to lack the recombinase function associated with RAG-1 and/or RAG-2 (e.g. commercially available TIM™ RAG-2 transgenic), or to lack Class I and/or Class II MHC antigens (e.g. the commercially available C1D and C2D transgenic strains). Of particular interest are mice that have a homozygous mutation at the scid locus, causing a severe combined immunodeficiency which is manifested by a lack of functionally recombined immunoglobulin and T-cell receptor genes. The scid/scid mutation is available or may be bred into a number of different genetic backgrounds, e.g. CB.17, ICR (outbred), C3H, BALB/c, C57BI/6, AKR, BA, B10, 129, etc. Other mice which are useful as recipients are NOD scid/scid; SGB scid/scid, bh/bh; CB.17 scid/hr; NIH-3 bg/nu/xid and META nu/nu.

As an alternative to reintroducing the genetically modified progenitor cells into an animal host, the cells may be introduced into a culture system capable of generating dendritic cells. For example, bone marrow cultures supplemented with Flt-3 ligand and cultured at high density have been shown to give rise to small lymphoid-sized cells, expressing CD11c, CD86, and major histocompatibility complex (MHC) class II (Brasel et al. (2000) Blood 2000 96(9):3029-3039), and having other characteristics of dendritic cells.

Genetic modification. The progenitor cells described above are genetically modified by the introduction of a cell growth promoting gene with expression targeted to dendritic cells via specific control sequences. Suitable vectors for modifying mammalian cells are well known in the art. The vectors may be episomal, e.g. plasmids, virus derived vectors such as cytomegalovirus, adenovirus, retrovirus, lentivirus, etc., or may be integrated into the target cell genome, through homologous recombination or random integration, e.g. retrovirus derived vectors such MMLV, HIV-1, ALV, etc. For examples of progenitor and stem cell genetic alteration, see Svendsen et al. (1999) Trends Neurosci. 22(8):357-64; Krawetz et al. (1999) Gene 234(1):1-9; Pellegrini et al. Med Biol Eng Comput. 36(6):778-90; and Alison (1998) Curr Opin Cell Biol. 10(6):710-5.

Cell growth promoting oncogenes are known in the art. Preferred oncogenes for the invention are strong oncogenes, which group includes simian virus 40 (SV40) large T antigen; polyoma virus middle T antigen or large T antigen; human papilloma virus E7 or E6 proteins; adenovirus E1A, v-ras; v-myc, v-fms, v-erbB, etc. In a preferred embodiment the oncogene is SV40 large T antigen.

The oncogene is operably linked to a conditional promoter that directs expression in dendritic cells at a sufficiently high level to allow generation of dendritic cells that can be maintained in culture, e.g. in the presence of suitable growth factors. Preferred promoters are selective for dendritic cells, where the level of expression in non-dendritic cells is sufficiently low that the animals or cell lineages can be maintained for a period of time sufficient to generate dendritic cells. That is, the oncogene is not so widely expressed that growth of the animal or cell lineage is not fatally disrupted.

Optimal promoters are derived from genes expressed strongly, and optionally selectively, in dendritic cells. Such promoters include the promoter from the gene encoding CD11c (see Lopez-Cabrera et al. (1993) J Biol Chem. 268(2):1187-93); from the genes encoding CD1 proteins, particularly CD1a (see Blumberg et al. Immunol Rev. 147:5-29); from the genes encoding MHC class II antigens, e.g. human HLA-DR, mouse IA alpha or beta genes, etc. (see Glimcher and Kara (1992) Annu Rev Immunol. 10:13-49); from the gene encoding CD86 (see Li et al (2000) Hum Immunol. 61(5):486-98; from the genes encoding cytokine receptors such as interleukin-3 receptor alpha (see Miyajima et al. (1995) Blood 85(5):1246-53); promoters from the genes encoding dendritic cell markers 33D1, CD83, CLEC, DCIR, DCL1, DC-SIGN, DEC205, Dectin, Langerin, MARCO, TLR3; and the like. In a preferred embodiment the promoter is the CD11c promoter.

Expansion of dendritic cells. The overall number of functionally mature dendritic cells in a host are optionally expanded through the prior administration of a suitable growth factor. For example, flt3-L has been found to stimulate the generation of large numbers of functionally mature dendritic cells, both in vivo and in vitro (U.S. Ser. No. 08/539,142, filed Oct. 4, 1995). Flt3-L refers to a genus of polypeptides that are described in EP 0627487 A2 and in WO 94/28391, both incorporated herein by reference. A human flt3-L cDNA was deposited with the American Type Culture Collection, Rockville, Md., USA (ATCC) on Aug. 6, 1993 and assigned accession number ATCC 69382. Other useful cytokines include granulocyte-macrophage colony stimulating factor (GM-CSF; described in U.S. Pat. Nos. 5,108,910, and 5,229,496 each of which is incorporated herein by reference). Commercially available GM-CSF (sargramostim, Leukine®) is obtainable from Immunex Corp., Seattle, Wash.) Moreover, GM-CSF/IL-3 fusion proteins (i.e., a C-terminal to N-terminal fusion of GM-CSF and IL-3) may be used. Such fusion proteins are well known in the art and are described in U.S. Pat. Nos. 5,199,942; 5,108,910 and 5,073,627, each of which is incorporated herein by reference.

Various routes and regimens for delivery may be used, as known and practiced in the art. For example, where the agent is Flt3-L, the Flt3-L may be administered daily, where the dose is from about 1-5000 μg/kg body weight, usually from about 10-1000 μg/kg body weight, and more usually from about 50-500 μg/kg body weight. Administration may be at a localized site, e.g. sub-cutaneous, or systemic, e.g. intraperitoneal, intravenous, and may include sustained delivery methods, e.g. osmotic pumps or microparticles (the latter is described in U.S. Pat. No. 6,120,807 “Prolonged release of GM-CSF”).

In vitro culture. The cells of the invention are isolated from a source of dendritic cells, which tissue may be fetal, neonatal, juvenile or adult. Any tissue source of progenitor, immature or mature dendritic cells can be used, including blood, bone marrow, brain, eye, heart, intestine, kidney, liver, lung, lymph node, skin, spleen, and thymus. Exemplary tissues for use as a source of the cells are spleen or bone marrow tissues. The unseparated cells may be plated in culture, or an isolation step to enrich for dendritic cells may be employed. An enriched cell population may be about 75% cells of the selected phenotype, more usually at least 90% cells of the selected phenotype. The enriched cell populations are separated from other cells, e.g. lymphocytes, etc., on the basis of specific markers on the cell surface, which markers are identified with affinity reagents, e.g. monoclonal antibodies; or on the basis of differential density after gradient centrifugation. After isolation or enrichment, or after an initial period of culture, the cells are preferably cloned, e.g. by colony formation in semi-solid media, limiting dilution, automated cell deposition, etc.

The selection of culture medium to isolate cells of the present invention or maintain these cells or their progeny is a matter of routine experimental design and within the ordinary skill in the art. At a minimum, culture media contain a carbon source, a nitrogen source, essential amino acids, vitamins and minerals. Media may be protein- or serum-free or may contain such components as growth factors or serum, as required. A preferred growth medium for the dendritic cells of the present invention is McCoy's media supplemented with 10% FBS, essential and non essential amino acids, vitamins, 2-ME, penicillin, streptomycin, L-glutamine, Na pyruvate and HEPES buffer.

Additional growth factors are added to the medium, preferably one or more of Flt3-ligand at a concentration of from about 50-1000 ng/ml; TNF-alpha; preferably at 5-500 U/ml and GM-CSF at a concentration of from about 5 to 100 ng/ml. Optional other factors that may be added to the culture medium include, but are not limited to: granulocyte colony-stimulating factor (G-CSF; preferably at about 25-300 U/ml), monocyte-macrophage colony-stimulating factor (M-CSF; preferably at about 100-1000 U/ml), IL-1α preferably at about 1-100 LAF units/ml), IL-1β (preferably at about 1-100 LAF units/ml), IL-3 (preferably at about 25-500 U/ml), IL-4 (preferably at about 50-100 ng/ml), IL-6 (preferably at about 10-100 ng/ml), IL-15 (preferably at about 50-500 ng/ml), stem cell factor (SCF; preferably at about 10-100 ng/ml), leukemia inhibitory factor (LIF; preferably at about 1-100 ng/ml), oncostatin M (OSM; preferably at 1-1000 ng/ml), transforming growth factor-beta-1 (TGF-beta1; preferably at about 0.1-10 ng/ml and thrombopoietin (TPO; preferably at about 1000-10,000 U/ml). Other optional agents that may be added to the culture medium include antibodies (e.g. anti-IFNα), natural or engineered soluble molecules (e.g. soluble receptor of nuclear factor-kappa B ligand; sRANKL), peptides (e.g. Fas-derived peptides), antisense oligonucleotides (e.g. anti-Bax or anti-MyD88), etc. to block the activity of antiproliferative, pro-apoptotic or differentiation-modifying factors endogenous to the cultures.

Transformation for growth factor independence, resistance to cell death and/or resistance to senescence. Cells that can be continuously cultured and do not die after a limited number of cell generations are referred to as “immortalized”, in contrast to finite primary cultures. Immortalization of cells can occur spontaneously or be chemically or virally induced. The dendritic cell lines of the invention may optionally be selected for, or transformed to be, growth factor independent, resistant to cell death, resistant to dedifferentiation and/or resistant to senescence.

Immortalization may be associated with transformation and significant changes in phenotype. The altered ability to be continuously cultured may be due to, for example, a deletion or mutation in one or more of the genes whose products play a role in cell division, senescence or death, or overexpression or mutation of one or more oncogenes that override the action of the division, senescence or death genes. Genes that may be transfected or transduced into the cells to enhance immortalization and growth factor independence include polyoma middle T antigen, adenovirus E1A, myc oncogenes, v-rel fusions, and the E6 and E7 genes of human papilloma virus type 16 or 18. Genes that may be transfected or transduced into the cells for resistance to senescence include telomerase. Genes that may be introduced for resistance to programmed cell death include the anti-apoptotic proteins in the bcl-2 family. Vectors suitable for introduction of such genes, and methods for their use, are known in the art.

Characterization of dendritic cell lines. Dendritic cells have functional activities and phenotypes that are specifically associated with the maturity of the cell. Cell surface markers useful in the characterization of and classification of dendritic cells include: CD11a; CD11b; CD11c; F4/80; Fcγ RII/III receptor (FcR); MHC class I; MHC class II; CD80; CD86; CD54; CD40; and CD117. The cell lines of the present invention have several phenotypic characteristics of mature dendritic cells including expression of CD11c, CD54, CD86, and MHC class II. The majority of the lines also express CD40.

Analyses of the cell surface using monoclonal antibodies are made using a flow cytometer (for a review of methods, see Viedma Contreras (1999) Clin Chem Lab Med. 37(6):607-22; Maino and Picker (1998) Cytometry 34(5):207-15; Drouet and Lees (1993) Biol Cell. 78(1-2):73-8. Briefly, the cells are either combined with monoclonal antibodies directly conjugated to fluorochromes, or with unconjugated primary antibody and subsequently with commercially available secondary antibodies conjugated to fluorochromes. The stained cells are analyzed using a flow cytometer (for example as available from Becton Dickinson, Mountain View, Calif.). Additional phenotypic characteristics of dendritic cells can be characterized by gene expression profiling e.g. by reverse-transcriptase polymerase chain reaction (RT-PCR). The cell lines of the present invention have several characteristics of dendritic cells including expression of CCR1, CCR5, and CXCR4. The majority of the lines also express CCR7.

Identification of cell lines having the phenotype of mature dendritic cells is confirmed by the cells' ability to stimulate the proliferation, cytokine production or cytotoxic activity of naïve CD8⁺ and/or CD4⁺ T cells in a MLR or proliferation assay or cytotoxicity assay, e.g. as described in the examples. Briefly, dendritic cell lines, with or without prior activation, are incubated with naïve T cells in the presence of antigen. In order to provide a more uniform T cell signal, the naïve T cells may be obtained from a transgenic animal having a T cell receptor transgene with a defined specificity. The ability of the dendritic cells to stimulate these T cells to differentiate into proliferating, cytokine-producing and/or cytotoxic T cells, i.e. differentiated or activated cells, in an antigen-specific manner is then measured.

Uses of dendritic cell lines. The subject cultured cells may be used in a wide variety of ways, e.g. in gene discovery, the generation of dendritic cell subset-specific antibodies and probes; defining stages of dendritic cell differentiation and maturation; for induction of antigen specific responses in T and/or B lymphocytes; for dissection of antigen specific anergy or tolerance in T and/or B lymphocytes; for characterization of dendritic cell-natural killer cell interactions; for in vitro and in vivo analyses of dendritic cell-endothelial cell interactions; and for screening assays.

The subject cells are useful for in vitro assays and screening to detect factors that are active on dendritic cells. A wide variety of assays may be used for this purpose, including immunoassays for protein binding; determination of cell growth, differentiation and functional activity; production of hormones; and the like.

Of particular interest is the examination of gene expression in the dendritic cells of the invention. The expressed set of genes may be compared with a variety of cells of interest, e.g. hematopoietic stem cells, dedicated myeloid progenitor cells, immature dendritic cells, etc., as known in the art. For example, in order to determine the genes that are regulated during development, one could compare the set of genes expressed at different stages during differentiation.

Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of particular mRNAs in dendritic cells is compared with the expression of the mRNAs in a reference sample, e.g. differentiated cells.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. Nos. 5,776,683; and 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. Nos. 5,134,854, and 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with arrays are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

In another screening method, the test sample is assayed at the protein level. Methods of analysis may include 2-dimensional gels; mass spectroscopy; analysis of specific cell fraction, e.g. lysosomes; elution of processed peptides from MHC antigens; and other proteomics approaches.

Diagnosis can be accomplished using any of a number of methods to determine the absence or presence or altered amounts of a differentially expressed polypeptide in the test sample. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

Conditioned medium, i.e. medium in which cells of the invention have been grown for a period of time sufficient to allow secretion of soluble factors into the culture, may be isolated at various stages and the components analyzed for the presence of factors secreted by the dendritic cells. Separation can be achieved with HPLC, reversed phase-HPLC, gel electrophoresis, isoelectric focusing, dialysis, or other non-degradative techniques, which allow for separation by molecular weight, molecular volume, charge, combinations thereof, or the like. One or more of these techniques may be combined to enrich further for specific fractions.

The dendritic cells may be used in conjunction with the culture system in the isolation and evaluation of factors associated with the differentiation and immune response of lymphocytes. Thus, the dendritic cells may be used in assays to determine the effect of antigen presentation and MHC class I and class II recognition, on lymphoid cells through analysis of the cell behavior, gene expression, etc. and/or to induce an antigen specific response. Dendritic cells of the invention may be “loaded” with antigen, e.g. a tumor antigen, in order elicit a specific T cell mediated immune response, either in vivo or in vitro.

The dendritic cells of the present invention can be advantageously used in antigen-specific lymphocyte activation assays. To generate activated dendritic cells, it is preferred that antigen be incubated with the dendritic cells for about 1 to 48 hours. The time required for endocytosis, processing and presentation of antigen is dependent upon the proteinaceous antigen being used for this purpose. Methods for measuring antigen uptake and presentation are known in the art. For example, dendritic cells can be incubated with a soluble protein antigen for 3-24 hours then washed to remove exogenous antigen.

These antigen-presenting stimulator cells are then mixed with responder cells, usually either naïve or primed CD8⁺ and/or CD4⁺ T lymphocytes. After an approximately 4-72 hour incubation (for primed T lymphocytes) or approximately 0.5-7 d period (for naïve T lymphocytes), the activation of T cells in response to the processed and presented antigen is measured. Typical assays include measurements such as T cell proliferation in response to antigen, or measurements of T cell mediated, antigen specific killing. Responder cell activation can also be measured by the production of cytokines, such as IL-2, or by determining T cell-specific activation markers via flow cytometry or by assaying gene expression changes via RT-PCR.

Alternatively, activated dendritic cells can be used to induce non-responsiveness in CD8⁺ or CD4⁺ T lymphocytes. In addition to MHC class I or class II recognition, T cell activation requires co-receptors on the antigen-presenting cell. By blocking or eliminating stimulation of such co-receptors, e.g. CD80, CD86, presentation of antigen by co-receptor-deficient dendritic cells can be used to render CD8⁺ or CD4⁺ T lymphocytes non-responsive or anergic or altered in their cytokine responses to antigen. Cell contact-dependent “tolerogenic” induction of such T cells can also be actively promoted if dendritic cells are induced or engineered to express ligands for molecules such as Notch or OX2R to preferentially elicit T-regulatory cells or Th2 cells. Tolerogenic induction may also be mediated by soluble factors released from dendritic cells such as IL-10 and/or IL-12p40 homodimer, arginase, nitric oxide (NO) or TGF-beta1. Dendritic cells may be selected, activated and/or modified to preferentially produce such factors before or during their interaction with T-cells. As well as production of soluble factors, mature dendritic cells may be pretreated to “exhaust” or diminish key features, such as IL-12 production and/or CD40 signaling. Subsequent T-cell contact may then be followed by reduction in Th1 generation or skewing of T-cell differentiation from Th1 to Th2 (Langenkamp et al. (2000) Nat. Immunol. 1:311). The generation and use of tolerogenic dendritic cells is exemplified by these methods, but does not exclude other approaches based on alteration of dendritic cell differentiation or function.

The dendritic cell lines described herein may also be used to activate B lymphocytes directly in a T cell independent manner, to present antigen, proliferate, produce antibodies, and/or switch antibody isotypes. Such DC-modified B cells may then be used to increase, alter or decrease T cell responses through antigen-dependent or antigen-independent signals.

The dendritic cells of the present invention can also be used in assays of dendritic cell interactions with natural killer (NK) cells and endothelial cells. In the former instance, in addition to MHC class I and class II recognition, NK cells may be directly cytotoxic for some dendritic cell types and this susceptibility may be altered by dendritic cell expression of intracellular, surface and secreted molecules. Conversely, dendritic cells may also produce NK cell stimulating factors such as IL-12 and IL-15. In the latter instance, migratory dendritic cells may modulate the functions of endothelial cells and the extracellular matrix surrounding them, while endothelial transmigration may have a differentiative influence on the dendritic cell that affects its activation status, survival, and/or the quality of its interactions with other cells.

Genes may be introduced into the dendritic cells for a variety of purposes. Alternatively, vectors are introduced that express antisense mRNA or ribozymes, thereby blocking expression of an undesired gene. Various techniques known in the art may be used to transfect the target cells, e.g. electroporation, calcium precipitated DNA, fusion, transfection, lipofection and the like. The particular manner in which the DNA is introduced is not critical to the practice of the invention. Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc.

The cells of the present invention may also be used for screening biological response modifiers, i.e. compounds and factors that affect the various metabolic pathways of dendritic cells. For example, the subject cells may be used to screen for molecules that enhance or inhibit dendritic growth or differentiation or antigen presentation itself. Typically the candidate compound will be added to the dendritic cells, and the response of the dendritic cells monitored through evaluation of cell surface phenotype, functional activity such as ability to present antigen, patterns of gene expression, and the like. Of particular interest are screening assays for agents that have a low toxicity for human cells.

The term “agent” as used herein describes any molecule, e.g. protein or pharmaceutical, with the capability of altering the phenotype of a dendritic cell. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, including polypeptides, nucleic acids, and small organic compounds, e.g. having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In addition, the cells of the present invention may be used to generate antibodies for cell-specific proteins. For example, antibodies to cell-surface markers may be generated and used to purify a subpopulation from a heterogenous population of cells using a cell sorting system. Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation. Phage display is particularly useful for poorly immunogenic cell surface molecules and can be conducted using subtraction-based approaches to preferentially select for molecules expressed by, for example, either activated or tolerogenic dendritic cells.

Because dendritic cells can take up, process and present exogenous antigen (including proteins, glycoproteins and peptides), these cells are valuable tools that can be used to identify dominant epitopes of a particular antigen. Dendritic cells naturally process and present exogenous protein, permitting epitope mapping studies that better mimic the in vivo process.

The dendritic cells of the present invention provide a stable, reproducible, homogeneous population of cells that can be cultured and obtained in significant numbers. The low frequency of dendritic cells in mononuclear cell preparations has hampered molecular, biochemical and physiological study of this unique cell. The cell lines permit an examination of patterns of gene expression in dendritic cells, as well as the molecules and processes, that enable antigen presentation. Cell components and their interaction with molecules and processes involved in antigen uptake, processing and presentation can be dissected. Furthermore, this homogeneous dendritic cell line can be used as an immunogen to identify lineage-specific markers for dendritic cells.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

### Experimental

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### Example 1

Construction of a Transgenic Mouse

Construction of an expression vector for SV40 large T antigen (SV40T-Ag) under the control of the murine CD11c promoter. The plasmid pBS5.3kb CD11c promoter and beta-globin cassette, containing ˜5.3 kb of murine CD11c 5′ flanking genomic DNA sequence linked to 3′ sequences from rabbit beta-globin cDNA, is described by Brocker et al. (1997) J. Exp. Med. 186:541; and Riedinger et al. (1997) Immunol. Lett. 57:155.

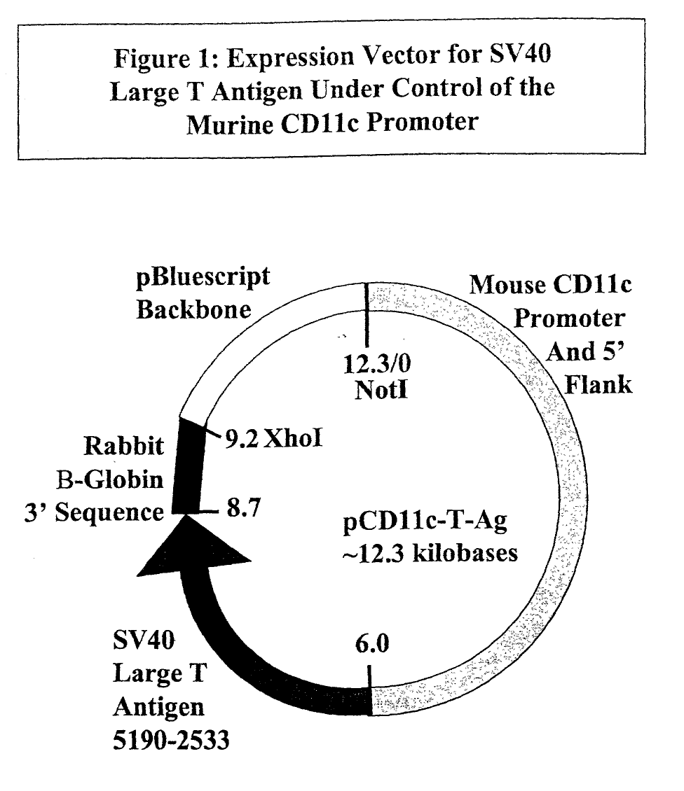

The SV40 early region, encoding small and large T-antigens (T-Ag) and containing sequences between Stul at SV40 map position 5190 and BamHI at position 2533, was cloned as a blunt fragment into the blunted EcoRI site of pBS5.3kb CD11c promoter+beta-globin to generate pCD11c-T-Ag. A map of the resulting construct is shown in FIG. 1.

The Bluescript backbone, CD11c regulatory sequence and rabbit beta-globin intron plus polyadenylation sequences are all described in the Brocker et al, supra. The SV40 T-ag sequence (5190-2533) is as described by Fiers et al. (1978) Nature 273 (5658):113-120.

Mice. CD11c-T-ag sequences were excised from the vector backbone by NotI+Xhol digestion, resolved and purified by gel electrophoresis followed by NA45 fragment isolation and injected into the male pronucleus of fertilized C57L/6×DBA/2 F2 eggs using established transgenic methodology. Injected eggs were transferred to pseudopregnant Swiss Webster females using established transgenic methodology and allowed to develop to term.

Mice carrying the transgene were identified using a PCR assay specific for SV40 sequences as follows. Ear biopsy genomic DNA was prepared using established procedures and amplified using the primers (SEQ ID NO:1) 5′-gga act gaa tgg gag cag tgg and (SEQ ID NO:2) 5′-gca gac act cta tgc ctg tgt gg, specific for a 381 bp sequence within SV40.

Mice were observed to develop “big head”: hydrocephalus secondary to choroid plexus papilloma formation, which has been associated with SV40 T-ag expression in transgenic mice (see Palmiter et al. (1985) Nature 316:457; Van Dyke et al. (1987) J. Virol. 61:2029.

### Example 2

### Isolation and Culture of Dendritic Cells and Dendritic Cell Precursors from Bone Marrow and Spleen

Cells from mice and initial culture conditions. Spleen and bone marrow tissues were harvested from 22 day old CD11c/SV40 transgenic mice bearing overt head tumors. Single cell suspensions were made from these tissues, red blood cells were lysed, and the remaining intact leukocytes were washed.

Washed cells were cultured in either 20 ng/ml GM-CSF alone, 20 ng/ml each GM-CSF+IL-4, or 100 ng/ml Flt3-ligand in McCoy's media supplemented with 10% FBS, essential and non essential amino acids, vitamins, 2-ME, penicillin, streptomycin, L-glutamine, Na pyruvate and HEPES buffer. Cells cultured in Flt3-ligand were seeded at 10⁶ cells/ml in 15 ml in 10 cm diameter petri dishes, while cells cultured in GM-CSF were seeded at 0.5 million cells/ml.

Passaging techniques and cell line generation. Adherent and non-adherent cells were passaged after 3 days and GM-CSF+IL-4 cultures were changed to media supplemented with GM-CSF but without IL-4. Cultures supplemented with Flt3-ligand were maintained with this supplementation at this time. Adherent and non-adherent cells were divided again on day 11. Cultures were divided again on days 21 and 25. After 25 days, on the basis of cell culture viability, supplementation of all cultures with Flt3-ligand was terminated and replaced thereafter by supplementation with GM-CSF. Also from this time on, only non-adherent cells were passaged. Cells were split every 7-9 days at a ratio of 1:2 or 1:4, and have been maintained in culture for >540 days.

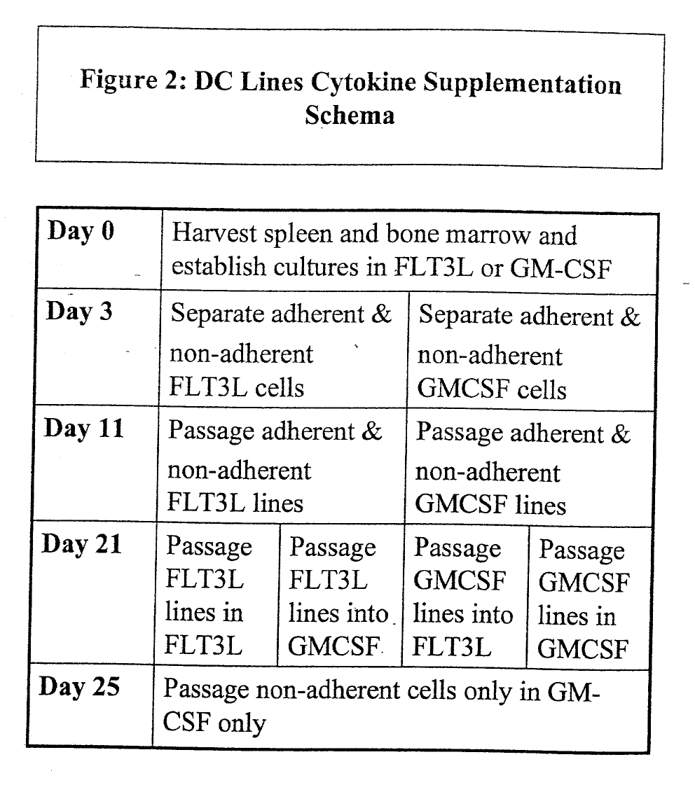

FIG. 2 is a flow chart depicting examples of cytokine combinations for culture.

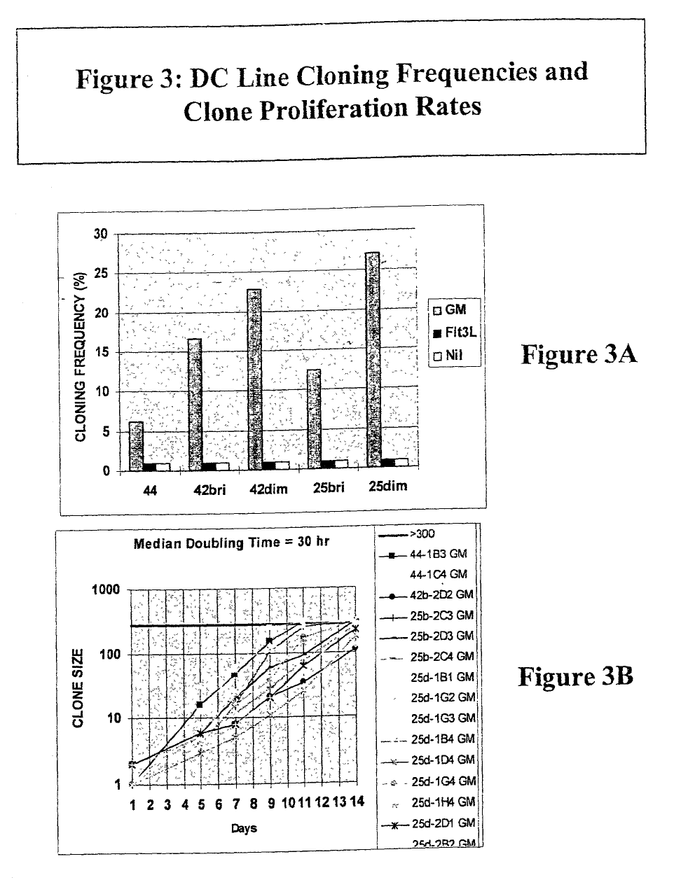

All cell lines were then cloned by limiting dilution and/or FACS with automated cell deposition. Cloning frequencies and clonal growth characteristics varied between different cell lines and between subclones, but all of the subclones generated to date were dependent on exogenous GM-CSF for continued growth. FIGS. 3A and 3B provide cloning efficiency and clone development information for different cell lines.

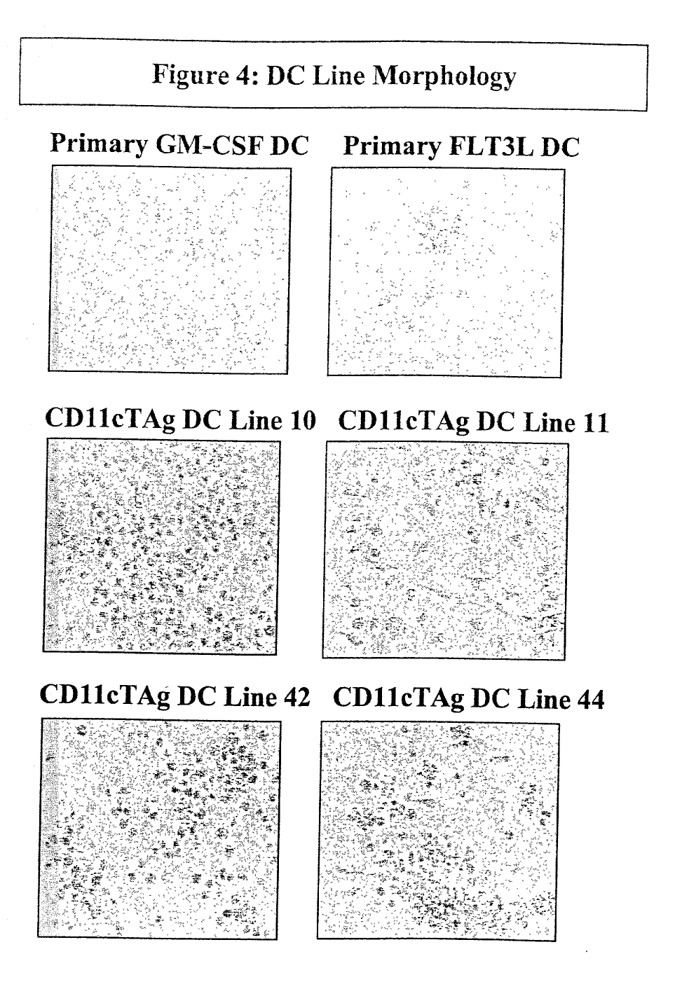

Analyses for dendritic cell characteristics. Non adherent cells with dendritic processes and clustering propensity were first observed on day 5 in the GM-CSF cultures and on day 18 in the Flt3-ligand cultures. Cell morphologies were first documented by photomicroscopy on day 25 and approximately monthly since then. FIG. 4 is a photograph depicting typical dendritic cell line morphology.

DC lines were first tested on day 21 for surface CD11c and CD11b by flow cytometry and have been tested for these and additional markers at least three times since then. Without exogenous stimulation, all the cell lines express CD11c, CD54, CD86, and class II MHC and varying levels of CD11b, CD40, CD80 and CD117. All the lines are negative for surface CD19, F4/80, B220, CD8, 33D1 and CD4 in the absence of stimulation. All the lines are positive for intracellular SV40 large T antigen. These results are consistent with dendritic cell phenotypes and not macrophage, granulocyte, NK, T lymphocyte or B lymphocyte phenotypes.

TABLE 1
DC Line panel surface antigen expression.

| Culture | | | | | | | | Class II |
|---|---|---|---|---|---|---|---|---|
| Designation | CD11b | CD11c | CD80 | CD86 | CD40 | CD54 | CD117 | MHC |
| 10 | Hi & Lo | + | nt | nt | nt | + | −/+ | + |
| 11 | + | + | + | + | + | + | + | + |
| 19 | Hi & Lo | + | < | + | < | + | + | + |
| 20 | Hi & Lo | + | < | + | < | + | < | + |
| 25 | + | + | < | + | + | + | + | + |
| 27 | Hi & Lo | + | < | + | + | + | + | + |
| 28 | + | + | + | + | + | + | + | + |
| 34 | Hi & Lo | + | + | + | + | + | −/+ | + |
| 36 | Hi & Lo | + | < | + | < | + | < | + |
| 37 | + | + | < | + | < | + | < | + |
| 42 | Hi & Lo | + | + | + | + | + | −/+ | + |
| 44 | Hi & Lo | + | < | + | + | + | < | + |
| 45 | Hi & Lo | + | < | + | + | + | −/+ | + |
| 53 | + | + | < | + | < | + | < | + |
| 59 | + | + | + | + | + | + | −/+ | + |
| 62 | Hi & Lo | + | + | + | + | + | + | + |

Hi & Lo = heterogeneous expression; + = high expression; −/+ = low expression; < = undetectable expression; nt = not tested.

### Example 3

### Functional Analysis of Dendritic Cell Lines

Most of the DC lines were screened for priming of T cell cytotoxicity and stimulation of T cell proliferation using either the DO11.10 T cell clone, ovalbumin-specific T cells from OT-I or OT-II TCR transgenic mice, or allogeneic T cells from normal C3H mice. Priming of cytotoxic T cell activity in the OT-I assay indicates successful class I MHC-mediated presentation of ovalbumin peptide by the DC lines to CD8+ T cells. Stimulation of T cell proliferation in the DO11.10 or OT-II assays indicate successful presentation and/or processing of ovalbumin protein or peptide by the DC lines to CD4+ T cells (see Daro et al. (2000) J. Immunol. 165:49). Most of the lines are competent for CTL priming and all of the tested lines are competent for stimulation of T cell proliferation in at least two assays. The long term clones derived from the parent lines are also competent for the stimulation of alloreactive T cell proliferation in a mixed leukocyte reaction.

The OTI priming experiments were performed as follows.

In vitro priming of naïve OTI CTL. This assay tests the ability of APC to prime and expand naïve CTL precursors. Dendritic cells are the best activators of CTL, macrophages display weak activity, and resting B cells and non-professional APC display no activity (see Sallusto & Lanzavecchia (1999) J Exp Med 189(4):611-4). The assay co-cultures dendritic cells, a peptide from ovalbumin, and CD8⁺ T cells from ovalbumin-specific TCR-transgenic mice, then measures the level of CD8-mediated cytotoxic activity generated (see Brossart & Bevan (1997) Blood 90(4):1594-1599; Clarke et al (2000) Immunol Cell Biol 78(2):110-7).

T cell priming. APC were prepared by coating with the OTI peptide: (SEQ ID NO:3) SIINFEKL (OTIp). At a concentration of ˜1 μM it is saturating, and a dose of ˜50 nM is typically in the linear portion of the CTL response to antigen dose at saturating numbers of DC. APC were titrated in 96 well U-bottom plates (10⁴-10²/well) and added to the TcR transgenic cells (10⁶ Spleen or LN cells/well, or 3×10⁵ purified CD8⁺ OTI cells). All points were set up in duplicate and included peptide-unpulsed DC controls. On days 1 and 2 half the tissue culture medium was exchanged. To assess priming a standard CTL assay was performed on day 3 of stimulation. The effector cells were titrated at an initial dilution of 1:1.5.

Effectors. The cells being tested for lytic activity (effector cells) were tested against both specific targets (antigen pulsed or cells that express the target antigen) and non-specific targets (unpulsed target cells or cells, preferably a parental cell line or some other cell line very closely related to the Ag+ target cells). The effectors were serially diluted against a constant number of target cells per well. Steps for the dilutions varied depending on circumstances (1:2-1:10) but for most assays dilutions of 1:3 were used: 150 μl of the effectors were added at the desired concentration to the top well, and carried 50 μl down into the next row of wells containing 100 μl of medium (all wells had 100 μl/well after carrying out the dilutions).

Target Cells. The target cells were peptide coated cultured cell lines. An alternative was cells transfected with the antigen of interest. Cultured target cells were growing well and not overgrown. Target cell lines include EL-4 and C1498 (H-2^{b}) and P815 (H-2^{d}).

Procedure. The targets were counted (using 1×10⁶/plate for the CTL assay) and resuspended at ˜5·10⁶/ml in medium. The effector cells were tested against Ag+ and Ag− targets. For the Ag+ targets peptide was added (normally to a final concentration of 1 μM peptide). To both sets of targets was added 50 μl/1×10⁶ cell of Na₂⁵¹CrO₄. They were incubated for 1-2 h at 37° C./7% CO₂ with the lids of the tubes loosened, then washed 3× with PBS. In an optional step to reduce spontaneous lysis: cells were incubated in 10 ml medium at 37° C. for 15-30 min, and then spun down (this allows the dead/dying/damaged cells that contribute most to background to leak their ⁵¹Cr before the assay starts). The medium was brought to a volume of 1×10⁵/ml and added to the assay on top of diluted effector cells at 100 μl/well.

Controls were set up at same time as the rest of assay:

spontaneous lysis: added 100 μl target cells to 100 μl medium

total lysis: added 100 μl target cells to 100 μl 1% Triton-X100 in water (with 1:500 green food color) or alternatively to 100 μl 1N HCI. The assay was incubated for 4 h at 37° C./7% CO₂. Plates were spun at 1200 rpm for 5 min; and harvested by transferring 100 μl supernatant to glass tubes in racks. Activity was measured in a γ-counter and calculated as % specific lysis using the formula:

% specific lysis=100×(sample cpm−target spontaneous cpm) (target total cpm−spontaneous cpm)

The protocols for the proliferation assays are described in Daro et al. (2000) J. Immunol. 165:49. The data for DC line activity in T cell proliferation and CTL priming assays are shown in Table 2.

TABLE 2
DC Line Activities in T Cell Proliferation and CTL Priming Assays

| DC Line | Proliferation Induction | CTL Priming |
|---|---|---|
| 10 | nt | < |
| 11 | nt | + |
| 19 | nt | + |
| 20 | nt | + |
| 25 | + | + |
| 27 | + | −/+ |
| 28 | nt | < |
| 34 | + | + |
| 36 | + | + |
| 37 | nt | < |
| 42 | + | −/+ |
| 44 | + | + |
| 45 | + | + |
| 53 | nt | + |
| 59 | nt | + |
| 62 | + | + |

+=significant activity; −/+ low activity; <=undetectable activity; nt = not tested.

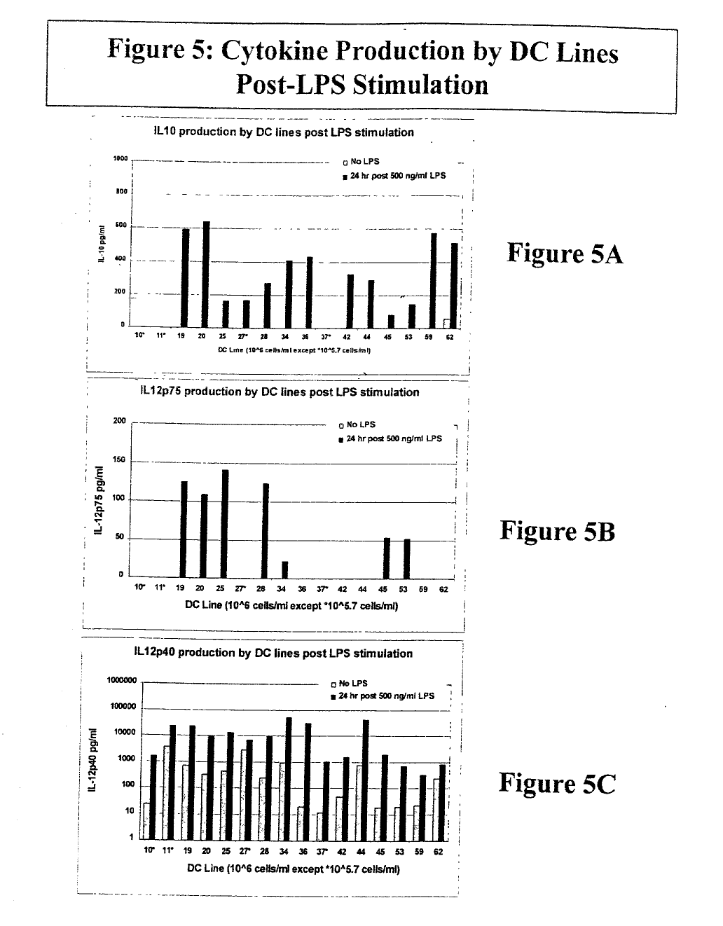

Response to stimulus. The DC lines were screened for secretion of IL10 and IL-12p40 and IL-12p70 in response to LPS (500 ng/ml for 24 hr). Cytokine secretion was assayed by ELISA using commercial kits as recommended by the manufacturers. Assay sensitivity was <50 pg/ml for IL-10 and IL-12p70; and was <20 pg/ml for IL-12p40. None of these cytokines were secreted in significant levels by any of the DC lines in the absence of stimulation. IL-10 secretion was variably inducible in all the lines, while IL-12p70 secretion was induced in about half of the DC lines. In all but one of the lines where IL-12p70 was undetectable, high levels of IL-12p40 were secreted. There was a trend towards an inverse relationship between the secreted levels of IL10 and IL-12p70. The data for constitutive and inducible IL-10, IL-12p70 and IL-12p40 secretion are shown in FIG. 5. Since IL-12p40 homodimers can act as antagonists of IL-12p70, and IL10 can inhibit the actions of IL-12p70, the lines secreting both IL-10 and IL-12p40 but not IL-12p70 may have tolerizing or Th2-skewing activities.

A subset of the DC lines were screened for constitutive expression of the following genes by Taqman PCR: IL-12p35, IL-12p40, IL6, IL10, 41BB, 41BBL, CD40, OX40, OX40L, RANK, RANKL, class II MHC, MyD88, CCR1, CCR3, CCR4, CCR5, CCR6, CCR7, CCR8, CCR9, CXCR3, CXCR4, and CXCR5. Each cell line had a unique pattern of expression of these genes with no perfect matches to the classical “immature” or “mature” DC phenotypes (as described by Sozzani et al. (1999) J. Leukoc. Biol. 66:1-9; and Vecchi et al. (1999) J. Leukoc. Biol. 66:489). However, the combination of these gene expression prolifes and the aforementioned FACS and functional data are consistent with a relatively “mature” DC phenotype. The data for gene expression patterns in provided in Table 3.

TABLE 3
Gene expression patterns of DC lines

| Gene | 10 | 20 | 36 | 42 | 44 | 59 | 62 |
|---|---|---|---|---|---|---|---|
| mb-actin | + | + | + | + | + | + | + |
| mPBGD | + | + | + | + | + | + | + |
| m41BB | nt | nt | nt | nt | + | nt | nt |
| mCD40 | nt | nt | nt | nt | + | nt | nt |
| mOX40L | nt | nt | nt | nt | + | nt | nt |
| mRANK | nt | nt | nt | nt | + | nt | nt |
| mMyD88 | nt | + | nt | nt | + | + | + |
| mIL-6 | + | + | < | + | + | + | + |
| mIL-10 | + | + | < | + | + | + | + |
| mIL-12p35 | + | + | < | < | < | + | + |
| mIL-12p40 | + | < | < | + | + | + | + |
| mCCR1 | + | + | + | + | + | + | + |
| mCCR3 | + | < | < | < | + | < | < |
| mCCR4 | + | < | + | + | + | + | < |
| mCCR5 | + | + | + | + | + | + | + |
| mCCR6 | + | < | < | + | + | < | < |
| mCCR7 | + | + | < | + | + | + | + |
| mCCR8 | < | < | + | + | < | < | < |
| mCCR9 | + | < | < | < | + | < | < |
| mCXCR3 | + | < | + | + | + | < | < |
| mCXCR4 | + | + | + | + | + | + | + |
| mCXCR5 | + | < | + | + | + | < | < |

+ = detectable; < = undetectable; nt = not tested.

Phenotypic and functional stability of cloned dendritic cell lines. Two of the parent cell lines, #44 and #62, were cloned by two rounds of limiting dilution and/or automated cell deposition to yield the clones 44hi2-15 and 62-1-6. These clones have been monitored for preserved expression of key constitutive and inducible dendritic cell phenotypic and functional characteristics. The characteristics of these two clones match those described for their parents in Tables 1-3 and FIGS. 4-5 with the exception that clone 62-1-6 can be induced to express IL-12p70 in response to stimuli other than LPS (see below). These and other key features have been retained for approximately 6-12 months since cloning and thus about 12-18 months since their respective parent lines were derived.

Analysis for viral infectivity. All the cell lines were tested for infectability with recombinant retroviruses and adenoviruses engineered for green fluorescent protein or beta-galactosidase reporter gene expression. The lines exhibited variable but significant infection rates with retroviruses (0-15%), and variable with medium infection rates with adenoviruses (5-50%). The retroviral infectivity levels are similar to or exceed those reported recently for the immature D1 mouse DC cell line (Gasperi et al. (1999) J. Leukocyte Biol. 66(2):263-267) and exceed those currently achievable for primary DC isolated from the spleens and lymph nodes of Flt3L- or GM-CSF-treated mice. In view of the relatively low level of infection of the DC lines with retro- and adeno-virus vectors, we examined whether alternatives such as lentivirus and herpesvirus vectors would yield improved infection rates. These vectors are less dependent on the receptor expression and replication status of target cells and may be less likely to perturb cell functions. The cloned line 44hi2-15 was tested for infectibility with a recombinant vesicular stomatitis virus pseudotyped lentivirus or a herpes simplex virus based herpesvirus engineered for green fluorescent protein expression. The results show that 40-50% infection can be achieved with standard stocks of both lentivirus and herpesvirus and that concentrated lentivirus stocks can be used to increase the multiplicity of infection to yield infection rates >90%. Importantly, the lentivirus infection levels were attained without significant cell activation or death and were maintained for at least 18 days post-infection. These data significantly improve the feasibility of virus-vector-based expression cloning using the DC lines as substrates for novel gene expression.

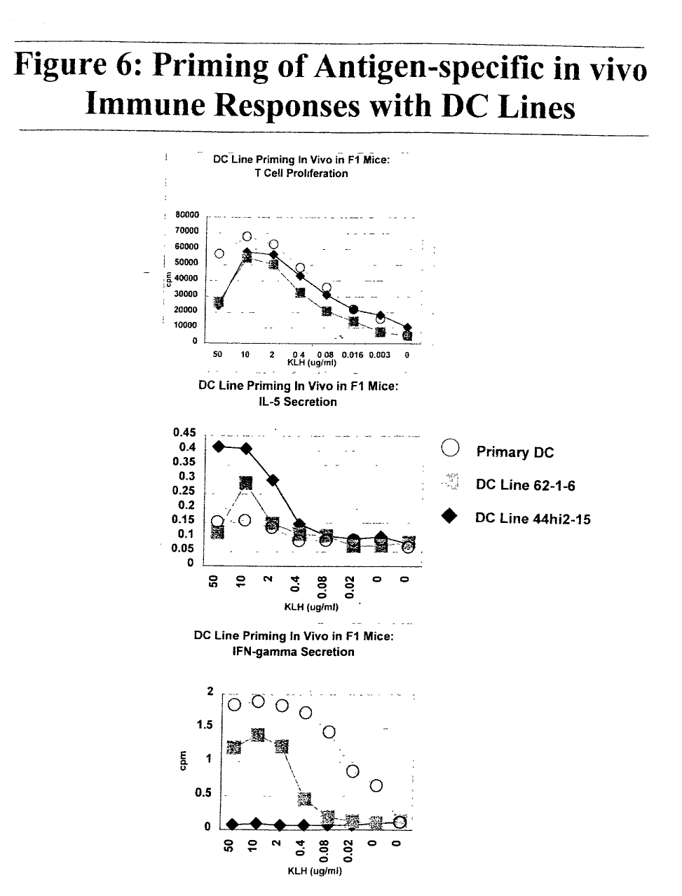

Priming of immune responses in vivo. The cloned DC lines 44hi2-15 and 62-1-6 were also tested for the ability to initiate antigen-specific immune responses in vivo. Cells were exposed to the protein antigen keyhole limpet hemocyanin (KLH) for 16 hr at 37 C., harvested, washed and adoptively transferred into the footpads of syngeneic C57BL/6-DBA/2 F1 mice. After 5 days, the draining popliteal lymph node cells were isolated, restimulated in vitro with KLH, and the proliferation and cytokine responses in the restimulation cultures were measured (FIGS. 6A, 6B and 6C). Both cloned DC lines elicited antigen-specific proliferative and IL-2 responses indicative of naïve T cell priming. Significantly, DC line 44hi2-15, which expresses IL-10 and IL-12p40 but not IL-12p70, also elicited high levels of IL-5 without detectable interferon-gamma (IFN-g): a Th2-polarized response. By contrast, DC line 62-1-6, which can be induced to express both IL-10 and IL-12p70 (see above), elicited significant levels of both IFN-g and IL-5: a non-polarized Th0 response. These results indicate that the DC lines are competent for protein antigen uptake and processing, migration in vivo from a peripheral tissue site to a lymphoid organ, stimulation of naïve T cell proliferation, and polarization of T cell cytokine responses. This adds to the utility of the lines in expression cloning scenarios using, for example, recombinant lentivirus libraries (see above) with in vivo selection of novel genes that modify dendritic cell and/or lymphocyte function. It also raises the possibility of using adoptive transfer of the DC lines to develop novel mouse models of disease, particularly, DC-initiated or -modulated models of infectious disease, cancer, transplantation, allergy or autoimmunity.

### Example 4

Regulation of IL-12p35 and IL-12p40 Gene Expression

The production of IL-12 in response to stimulation was further studied at the level of gene expression. IL-12p70 is produced as a heterodimer between the IL-12p40 and IL-12p35 subunits. As noted above, if a cell produces only IL-12p40 subunits, this subunit can homodimerize into a p80 protein that acts as an antagonist of the activity of IL-12p70.

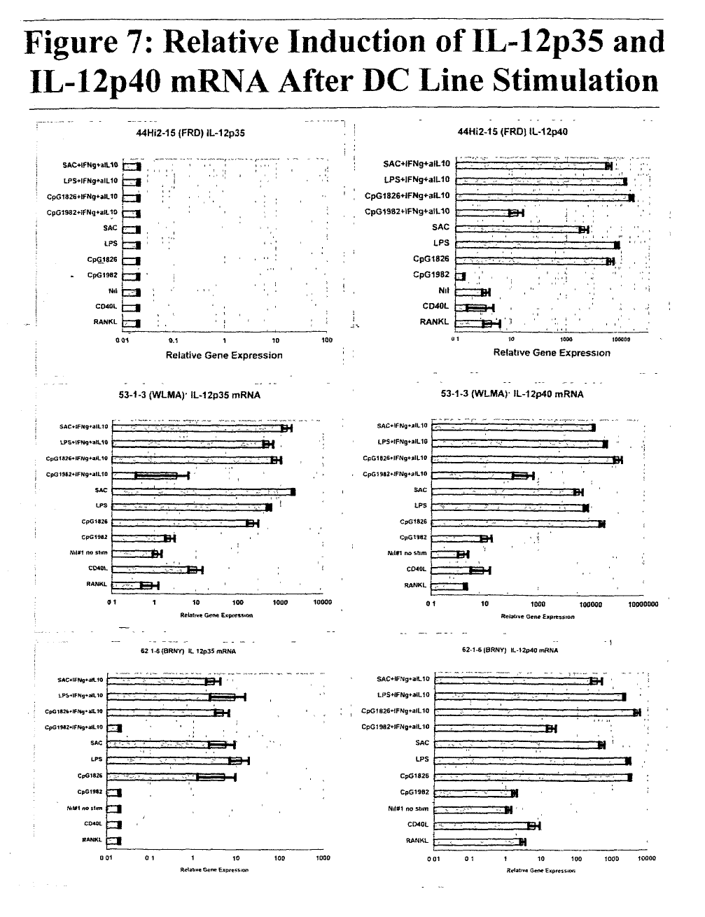

Three different subcloned DC lines were subjected to a variety of stimuli as indicated in FIG. 7. LPS treatment (500 ng/ml) was as described above. SAC (Staphyloccus aureus-formalin killed; 20 microgram/ml), IFNgamma (20 ng/ml), antilL-10 antibody (BD Pharmingen, Calif.; 5 microgram/ml), CpG1826 (1 microgram/ml) and CpG1982 (1 microgram/ml), muCD40L (1 microgram/ml) and RANKL (100 ng/ml) treatment were also used. Relative gene expression of the mRNAs encoding for the IL-12p40 and the IL-12p35 subunits after treatment was measured by Taqmane PCR.

As can be seen in FIG. 7, all three of the cell lines induced the gene encoding for the IL-12p40 subunit in response to most stimulatory factors tested. However, induction of the IL-12p35 subunit in response to stimulation differed between these three cell lines. One line, 44Hi2-15, did not product any IL-12p35 subunit message in response to any stimulation. This line is particularly useful for study because it has features similar to that of neonatal dendritic cells that produce only homodimer IL-12p80 antagonist. Antagonizing IL-12 receptor can drive T cells into a more TH2 response, and this cell line can be used to study this effect both in in vitro and in vivo assays of T cell activation. Moreover, such assays can form the basis for molecule discovery (e.g., molecules that interfere with induction of a TH2 response). In addition, this cell line is useful for identifying and studying interactions between new members of the IL-12 family, such as IL-23p19), because it lacks expression of a key IL-12 family member (IL-12p35).

The other two cell lines, 53-1-3 and 62-1-6, did induce expression of the gene encoding the IL-12p35 subunit in response to certain stimuli. Thus, these cell lines are capable of agonizing the IL-12 receptor and driving a TH1 response. The 62-1-6 cell line, in particular, demonstrated no resting expression of the IL-12p35 subunit, and very specific induction profile. Thus, this same line under different conditions would have an agonistic activity, or an antagonistic activity on an IL-12 receptor-expressing cell. Thus, cell lines such as these are also useful in both in in vitro and in vivo assays of T cell activation. Further, such assays can form the basis, alone or in combination with the IL-12 antagonistic cell lines, for molecule discovery.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of generating immortalized dendritic cells, the method comprising:
culturing dendritic cells obtained from a genetically modified progenitor cell, wherein said progenitor cell comprises a cell growth promoting oncogene under the regulatory control of a promoter that is active in dendritic cells, in medium comprising one or more of Flt-3 ligand and GM-CSF; and
maintaining said cultured dendritic cells for a period of at least 2 months;
wherein said dendritic cells express CD11c, and are capable of presenting antigen to stimulate naïve CD8⁺ and/or CD4⁺ T cells and/or B cells to an antigen specific response.

2. The method of claim 1, wherein said dendritic cells express CD86, CD54 and class II MHC antigens.

3. The method of claim 1, wherein said dendritic cells are obtained from a transgenic non-human animal comprising genetically modified progenitor cells.

4. The method of claim 1, wherein said genetically modified progenitor cell is a stem cell.

5. The method of claim 1, wherein said stem cell is transplanted into an animal host to provide for differentiation into dendritic cells.

6. The method of claim 1, wherein said stem cell is grown in culture to provide for differentiation into dendritic cells,

7. The method according to claim 3, wherein said stem cell is a human hematopoietic stem cell.

8. The method according to claim 1, wherein said cell growth promoting oncogene is a viral oncogene.

9. The method according to claim 7, wherein said viral oncogene is SV40 large T antigen.

10. The method according to claim 7, wherein said promoter that is active in dendritic cells is selectively expressed in dendritic cells,

11. The method according to claim 9, wherein said promoter is the CD11c promoter.

12. The method according to claim 3, wherein said transgenic animal is stimulated with Flt-3 ligand and/or GM-CSF prior to isolation of said dendritic cells or their precursors.

13. The method according to claim 1, wherein said cultured dendritic cells are selected or transformed for one or more of: growth factor independence; resistance to programmed cell death, resistance to dedifferentiation, and resistance to cell senescence.

14. The method according to claim 1, wherein said cultured dendritic cells are capable of migrating in vivo from a peripheral tissue site to a lymphoid organ.

15. An immortalized dendritic cell line, wherein said dendritic cells express CD11c, and are capable of presenting antigen to stimulate naïve CD8⁺ and/or CD4⁺ T cells, and/or B cells to an antigen specific response.

16. The immortalized dendritic cell line of claim 15, wherein said dendritic cells express CD54, CD86 and class II MHC antigens.

17. The immortalized dendritic cell line of claim 15, wherein said dendritic cells are obtained from a transgenic non-human animal comprising genetically modified progenitor cells.

18. The immortalized dendritic cell line of claim 15, wherein said genetically modified progenitor cell is a stem cell.

19. The immortalized dendritic cell line of claim 18, wherein said stem cell is transplanted into an animal host to provide for differentiation into dendritic cells.

20. The immortalized dendritic cell line of claim 19, wherein said stem cell is grown in culture to provide for differentiation into dendritic cells,

21. The immortalized dendritic cell line of claim 18, wherein said stem cell is a human hematopoietic stem cell.

22. The immortalized dendritic cell line of claim 15, wherein said cell comprises a cell growth promoting oncogene under the regulatory control of a promoter that is active in dendritic cells.

23. The immortalized dendritic cell line of claim 22, wherein said oncogene is SV40 large T antigen.

24. The immortalized dendritic cell line of claim 22, wherein said promoter that is active in dendritic cells is selectively expressed in dendritic cells.

25. The immortalized dendritic cell line of claim 24, wherein said promoter is the CD11c promoter.

26. The immortalized dendritic cell line of claim 15, wherein said cell line is derived from a transgenic animal stimulated with Flt-3 ligand and/or GM-CSF prior to isolation of said dendritic cells or their precursors.

27. The immortalized dendritic cell line of claim 15, wherein said cultured dendritic cells are selected or transformed for one or more of: growth factor independence; resistance to programmed cell death, resistance to dedifferentiation, and resistance to cell senescence.

28. The cultured dendritic cell of claim 15, wherein said cultured dendritic cells are capable of migrating in vivo from a peripheral tissue site to a lymphoid organ.

29. A method of screening a candidate biological response compound for its effects on dendritic cells, the method comprising:
contacting the immortalized dendritic cell of claim 15 with said candidate biological response compound; and
determining the effect on the dendritic cell phenotype.

30. The method according to claim 29, wherein said step of determining the effect on dendritic cell phenotype comprises analysis of cell morphology and cell surface expression of markers.

31. The method according to claim 29, wherein said step of determining the effect on dendritic cell phenotype comprises analysis of gene expression changes including the levels and organization of intracellular nucleic acids, proteins, lipids and carbohydrates and secreted extracellular molecules.

32. The method according to claim 29, wherein said step of determining the effect on dendritic cell phenotype comprises functional analysis of antigen uptake, processing, presentation or cell division.

33. The method according to claim 29, wherein said step of determining the effect on dendritic cell phenotype comprises functional analysis of in vivo migration from a peripheral tissue site to a lymphoid organ.

34. The method according to claim 29, wherein said step of determining the effect on dendritic cell phenotype comprises functional analysis of antigen presentation to stimulate naïve CD8⁺ and/or CD4⁺ T cells, and/or B cells to an antigen specific response

35. A method of inducing an antigen specific response in a T cell, the method comprising:
activating an immortalized dendritic cell according to claim 15 with said antigen and/or a non-specific stimulus to alter dendritic cell expression of cell surface and/or secreted molecules involved in intercellular interactions.

36. A method of obtaining nucleic acid sequences expressed in dendritic cells, the method comprising:
isolating mRNA from the immortalized dendritic cell of claim 15; and
obtaining any nucleic acid, protein, carbohydrate or lipid molecule.
